(19) United States
(12) Patent Application Publication
(10) Pub. No.: US 2009/0263449 A1
(43) Pub. Date: Oct. 22, 2009

(54) DELIVERY OF NUCLEIC ACID COMPLEXES FROM MATERIALS INCLUDING NEGATIVELY CHARGED GROUPS

(75) Inventors: Joseph Schmidt McGonigle, Minneapolis, MN (US); Aron Brent Anderson, Minnetonka, MN (US)

Correspondence Address: Pauly, Devries Smith & Deffner, L.L.C., Plaza VII, 45 South Seventh Street, Suite 3000, Minneapolis, MN, 55402-1630, US

(73) Assignee: SurModics, Inc., Eden Prairie, MN (US)

(21) Appl. No.: 12/421,285

(22) Filed: Apr. 9, 2009

Related U.S. Application Data
(60) Provisional application No. 61/043,505, filed on Apr. 9, 2008.

(51) Int. Cl. A61F 2/00; A61K 31/7088

(52) U.S. Cl. 424/423; 514/44 R; 514/44 A

(57) ABSTRACT

Embodiments of the invention include devices and methods for the controlled elution of nucleic acid delivery complexes. In an embodiment, the invention includes a medical device including a substrate surface, a polymeric coating disposed on the surface, the polymeric coating coupled to the substrate surface through the reaction product of a photoreactive group; the polymeric coating comprising negatively charged species on the surface; and a plurality of nucleic acid delivery complexes disposed on the polymeric coating, the nucleic acid delivery complexes comprising a nucleic acid and a cationic carrier agent complexed to the nucleic acid. Other embodiments are included herein.

This application claims the benefit of U.S. Provisional Application No. 61/043,505, filed Apr. 9, 2008, the contents of which are herein incorporated by reference.

### FIELD OF THE INVENTION

The present invention relates to devices and methods for the controlled release of active agents. More specifically, the present invention relates to devices and methods for the controlled release of nucleic acid delivery complexes.

### BACKGROUND OF THE INVENTION

One promising approach to the treatment of various medical conditions is the administration of nucleic acids as therapeutic agents. By way of example, this approach can include the administration of RNA, DNA, siRNA, miRNA, piRNA, shRNA, antisense nucleic acids, aptamers, ribozymes, catalytic DNA and the like.

In order to mediate an effect on a target cell, a nucleic acid based active agent must generally be delivered to an appropriate target cell, taken up by the cell, released from an endosome, and transported to the nucleus or cytoplasm (intracellular trafficking), among other steps. As such, successful treatment with nucleic acids depends upon site-specific delivery, stability during the delivery phase, and a substantial degree of biological activity within target cells. For various reasons, these steps can be difficult to achieve. As one example, nucleic acids are readily degraded by enzymes in the in vivo environment.

Accordingly, a need remains for devices that can deliver therapeutic nucleic acids to a target tissue and methods of making and using the same.

### SUMMARY OF THE INVENTION

Embodiments of the invention include devices and methods for the controlled elution of nucleic acid delivery complexes. In an embodiment, the invention includes a medical device including a substrate surface and a polymeric coating disposed on the surface. The polymeric coating can be coupled to the substrate surface through the reaction product of a photoreactive group. The polymeric coating can include negatively charged species on the surface. The device can include a plurality of nucleic acid delivery complexes disposed on the polymeric coating. The nucleic acid delivery complexes include a nucleic acid and a cationic carrier agent complexed to the nucleic acid.

In an embodiment, the invention includes a medical device including a polymer bound to a surface, the polymer having a net negative charge in an aqueous solution. The medical device can include a plurality of nucleic acid delivery complexes disposed on the surface of the polymer. The nucleic acid delivery complexes can be electrostatically held to the surface of the polymer. The polymer can be coupled to a substrate surface through the reaction product of a photoreactive group.

In an embodiment, the invention includes a medical device including a polymeric matrix comprising a polymer with anionic groups. The medical device can include a plurality of nucleic acid delivery complexes disposed within the polymeric matrix. The nucleic acid delivery complexes can include a nucleic acid and a carrier agent complexed to the nucleic acid.

In an embodiment, the invention includes a method of depositing a coating with an active agent on a medical device including depositing a polymer solution onto a substrate to form a coated substrate, exposing the coated surface to actinic radiation, and contacting the coated substrate with nucleic acid delivery complexes. The polymer solution can include a polymer with negatively charged species and photoreactive groups. The nucleic acid delivery complexes can include a nucleic acid and a carrier agent complexed to the nucleic acid.

In an embodiment, the invention includes a method of making an implantable medical device including depositing a polymer composition, modulating the density of negatively charged groups of the polymer composition, and disposing nucleic acid delivery complexes on the polymer composition. Modulation of the density of negatively charged groups of the polymer composition can result in control of the release of the nucleic acid delivery complexes from the polymer composition. The nucleic acid delivery complexes can include a nucleic acid and a cationic carrier agent complexed to the nucleic acid.

In an embodiment, the invention includes a method of controlling the release rate of nucleic acid delivery complexes including modulating the charge density of a drug delivery matrix. The drug delivery matrix can include a polymer and nucleic acid delivery complexes electrostatically held to the drug delivery matrix.

In an embodiment, the invention includes a method of providing controlled release of a nucleic acid including selecting a desired release profile and modulating the charge density of a drug delivery matrix according to the desired release profile.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a cross-sectional schematic view of a device in accordance with an embodiment.

FIG. 2 is a cross-sectional schematic view of a device in accordance with another embodiment.

FIG. 3 is a cross-sectional schematic view of a device in accordance with another embodiment.

FIG. 4 is a graph showing the amount of DNA that was bound to various surfaces in the form of nucleic acid delivery complexes.

FIG. 5 is a graph showing transfection of HEK-293 cells.

FIG. 6 is a graph showing the release of nucleic acid delivery complexes from various surfaces.

FIG. 7 is a graph showing luciferase expression in cells for various experimental conditions.

FIG. 8 is a schematic view of a medical device in accordance with an embodiment herein.

FIG. 9 is a cross-sectional schematic view of a medical device as taken along lines 9-9′ of FIG. 8.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

As used herein, the term “complex” shall refer to a chemical association of two or more chemical species through non-covalent bonds.

As used herein, the term “matrix” shall refer to a three-dimensional polymer network. In some embodiments, matrices can be disposed on a substrate, such as in the form of a coating over a substrate. In other embodiments, a substrate may be omitted (e.g., a matrix may be unsupported by a substrate).

One approach to maintaining the activity of nucleic acid-based therapeutic agents is to complex the nucleic acids with a delivery agent prior to administration to a mammalian subject. By way of example, nucleic acids (having a net negative charge) have been complexed with carrier agents having a net positive charge, such as polyethyleneimine, in order to prevent degradation during the delivery phase and enhance cell entry. These nucleic acid/carrier complexes are sometimes referred to as polyplexes or nucleic acid delivery complexes. While such an approach can aid in preserving the activity of the nucleic acid during the delivery phase, it does not address the issue of controlled release of the nucleic acid.

It is shown herein that one useful technique for administering nucleic acid delivery complexes involves disposing them on a surface or in a matrix wherein they are held in place through electrostatic interactions. Nucleic acid delivery complexes typically include positively charged groups on their exterior provided by the cationic carrier agent. As such, nucleic acid delivery complexes can be electrostatically held to other components, such as a surface or a matrix that include negatively charged groups. Because various materials can be readily modified to include negatively charged groups, this approach can be applied in the context of many different types of substrates and matrices.

Further, as shown herein, this approach can be used to modulate the active agent release characteristics of the resulting medical device. Depending on the specific medical application, it may be desirable to provide a faster or slower release rate of a nucleic acid active agent. Where a slower release rate is desired, the surface or matrix can be provided with additional negatively charged groups such that the nucleic acid delivery complexes are bound more tightly. Conversely, where a faster release rate is desired, the surface or matrix can be provided with fewer negatively charged groups such that the nucleic acid delivery complexes are bound less tightly.

One useful approach to providing negative groups on the surface of a broad range of substrates is to first select a polymer including a desired amount of negatively charged groups, or modify a polymer to include the same, and then couple the polymer to the surface of the substrate. In accordance with various embodiments herein, this approach can be accomplished through latent reactive chemistry such as photochemistry. For example, a polymer that includes negatively charged groups can also include one or more latent photoreactive groups. After the polymer is disposed on a desired substrate that includes carbon-hydrogen bonds, the photoreactive groups can be activated with actinic energy to form a reactive species that can then form covalent bonds with the substrate through the carbon-hydrogen bonds.

In some embodiments, polymers that include negatively charged groups can also include hydrolytically labile bonds. Such hydrolytically labile bonds can break within the in vivo environment such that the portion of the polymer including the negatively charged group will become dislocated. Such an approach can be used to further modulate the release rate of the nucleic acid delivery complexes.

Referring now to FIG. 1, a cross-sectional schematic view of a portion of a device 100 in accordance with an embodiment is shown. The device 100 can include a substrate 102. The substrate can include a variety of different materials including polymers, ceramics, metals, and the like. Examples of suitable substrates are provided in greater detail below. The surface 103 of the substrate 102 can include negatively charged groups 104. The surface can include a sufficient number of negatively charged groups to be capable of electrostatically bonding to nucleic acid delivery complexes. One technique for characterizing the number of negatively charged groups is by charge density. The charge density for the surface can be negative. Another technique for characterizing the number of negatively charged groups is to estimate the number of such groups over a given area. In some embodiments, the density of negatively charged groups is greater than about 0.1 micromoles per square meter. For example, the density can be greater than about 0.1 micromoles of SO₃ per square meter. In some embodiments, the density of negatively charged groups can be between 0.1 and 10 micromoles per square meter.

The device can include a plurality of nucleic acid delivery complexes 106 configured for release in vivo. The nucleic acid delivery complexes 106 can include positively charged groups on the outside surface of the complexes. The nucleic acid delivery complexes 106 can be electrostatically held to the substrate 102 through the negatively charged groups 104. Aspects of nucleic acid delivery complexes are provided below.

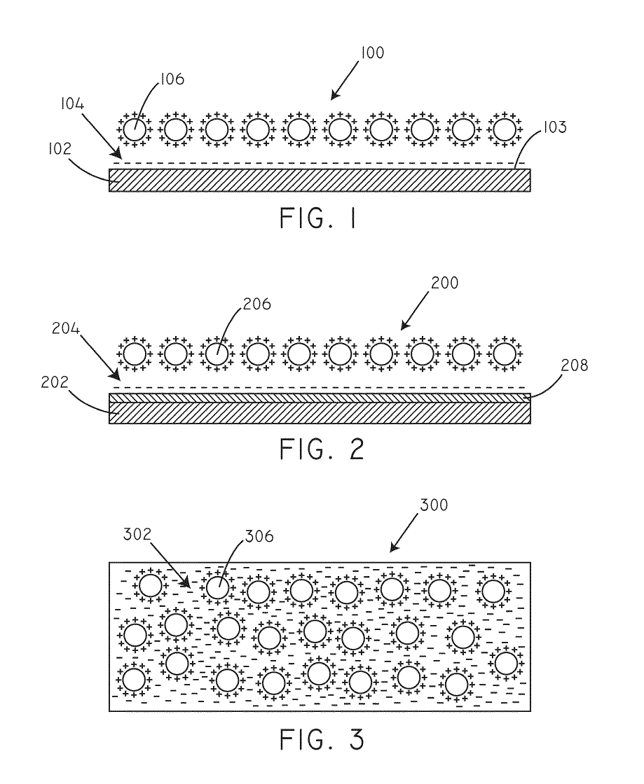

In some embodiments, a material layer can be provided over a substrate in order to provide negative groups on the surface for purposes of coupling with nucleic acid delivery complexes. For example, a layer of a polymer having negatively charged groups can be disposed over a substrate. Referring now to FIG. 2, a cross-sectional schematic view of a portion of a device 200 in accordance with an embodiment is shown. The device 200 can include a substrate 202 and a coating layer 208 disposed on the substrate 202. It will be appreciated that many different techniques can be used to apply the coating layer 208 onto the substrate 202. By way of example, techniques for applying such coating layers can include spray deposition, dip coating, brush coating, printing, and the like. In some embodiments, photoreactive groups can be used to couple the coating layer 208 to the substrate 202. Exemplary photoreactive groups and polymers that can include the same are described in greater detail below.

The coating layer includes negatively charged groups 204 on its surface. The coating layer can include any type of material that includes or can be modified to include negatively charged groups. A plurality of nucleic acid delivery complexes 206 can be electrostatically coupled to the substrate 202 through the negatively charged groups 204.

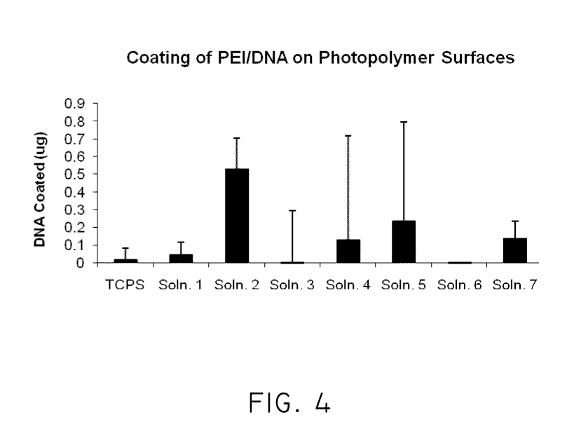

In some embodiments, a device can include a matrix with negatively charged groups distributed throughout the matrix and/or on the surface of the matrix. For example, a polymeric matrix can be formed with one or more polymers that include negatively charged groups. Referring now to FIG. 3, an embodiment of a device 300 including a matrix 302 with negatively charged groups is shown. The negatively charged groups are associated with the polymer(s) of the matrix and are distributed throughout the matrix. A plurality of nucleic acid delivery complexes 306 can be distributed throughout the matrix 302. The matrix can be deposited in various ways including spray deposition, dip coating, brush coating, printing, casting, and the like. In some embodiments, nucleic acid delivery complexes can be mixed in with polymers of the matrix before the matrix is formed. In other embodiments, a solution including nucleic acid delivery complexes can be contacted with a preformed matrix including negatively charged groups.

Various aspects of exemplary embodiments will now be described in greater detail.

### Nucleic Acid Delivery Complexes

Nucleic acid delivery complexes used in accordance with embodiments herein can include a nucleic acid as an active agent and a carrier agent complexed to the nucleic acid. In some circumstances, complexes of nucleic acids and carrier agents have also been referred to as “nucleic acid delivery particles”. For purposes of this application, the terms “nucleic acid delivery complex” and “nucleic acid delivery particle” shall be interchangeable.

Carrier agents used with embodiments of the invention can include those compounds that can be complexed with nucleic acids in order to preserve the activity of the nucleic acids during the manufacturing and delivery processes. Exemplary carrier agents can also include those effective to promote internalization of nucleic acids into cells. Exemplary classes of suitable carrier agents can include cationic compounds (compounds having a net positive charge) and charge neutral compounds. By way of example, suitable carrier agents can include cationic macromolecules such as cationic polymers. Suitable carrier agents can also include cationic lipids. Suitable cationic carrier agents can also include polycation containing cyclodextrin, histones, cationized human serum albumin, aminopolysaccharides such as chitosan, peptides such as poly-L-lysine, poly-L-ornithine, and poly-4-hydroxy-L-proline ester, peptides including protein transduction domains, and polyamines such as polyethylenimine (PEI), polypropylenimine, polyamidoamine dendrimers, and poly(beta-aminoesters). Other carrier agents can include solid nucleic acid lipid nanoparticles (SNALPs), liposomes, protein transduction domains and polyvinyl pyrrolidone (PVP). Additionally, carriers may also be conjugated to molecules which allow them to target specific cell types. Examples of targeting agents include antibodies and peptides which recognize and bind to specific cell surface molecules.

Nucleic acids used with embodiments of the invention can include various types of nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

Nucleic acid delivery complexes can be formed from carrier agents and nucleic acids through various processes. In some cases, for example, a cationic carrier agent interacts with an anionic nucleic acid molecule and condenses into a compact, ordered complex. As such, in some embodiments, the nucleic acid can simply be contacted with the cationic carrier agent in order to form nucleic acid delivery complexes.

The effective size of the nucleic acid delivery complexes can be affected by various factors including the molecular weight of the carrier agent, conditions during complex formation, and the molecular weight of the nucleic acid, amongst others. However, while not intending to be bound by theory, it can be desirable to control the size of the nucleic acid delivery complexes. For example, complexes that are excessively large may be much less effective at transfection than smaller complexes. In some embodiments, these complexes can have a diameter of less than about 1 micron. In some embodiments, these complexes can have a diameter of less than about 500 nm. In some embodiments, these complexes can have a diameter of less than about 200 nm. In some embodiments, these complexes can have a diameter of between about 20 and 200 nm.

### Photoreactive Groups

Some embodiments can include the use of compounds with photoreactive groups. The photoreactive groups can be utilized to couple polymer(s) having negatively charged groups to a surface or a substrate. As used herein, the phrases “latent photoreactive group” and “photoreactive group” are used interchangeably and refer to a chemical moiety that is sufficiently stable to remain in an inactive state (i.e., ground state) under normal storage conditions but that can undergo a transformation from the inactive state to an activated state when subjected to an appropriate energy source. Photoreactive groups respond to specific applied external stimuli to undergo active specie generation with resultant covalent bonding to an adjacent chemical structure. For example, in an embodiment, a photoreactive group can be activated and can abstract a hydrogen atom from an alkyl group. A covalent bond can then form between the compound with the photoreactive group and the compound with the C—H bond. Suitable photoreactive groups are described in U.S. Pat. No. 5,002,582, the disclosure of which is incorporated herein by reference.

Photoreactive groups can be chosen to be responsive to various portions of actinic radiation. Typically, groups are chosen that can be photoactivated using either ultraviolet or visible radiation. Suitable photoreactive groups include, for example, azides, diazos, diazirines, ketones, and quinones. The photoreactive groups generate active species such as free radicals including, for example, nitrenes, carbenes, and excited states of ketones upon absorption of electromagnetic energy.

In some embodiments, the photoreactive group is an aryl ketone, such as acetophenone, benzophenone, anthrone, and anthrone-like heterocycles (i.e., heterocyclic analogs of anthrone such as those having N, O, or S in the 10-position), or their substituted (e.g., ring substituted) derivatives. Examples of aryl ketones include heterocyclic derivatives of anthrone, including acridone, xanthone, and thioxanthone, and their ring substituted derivatives. Other suitable photoreactive groups include quinone such as, for example anthraquinone.

The functional groups of such aryl ketones can undergo multiple activation/inactivation/reactivation cycles. For example, benzophenone is capable of photochemical excitation with the initial formation of an excited singlet state that undergoes intersystem crossing to the triplet state. The excited triplet state can insert into carbon-hydrogen bonds by abstraction of a hydrogen atom (from a polymeric coating layer, for example), thus creating a radical pair. Subsequent collapse of the radical pair leads to formation of a new carbon-carbon bond. If a reactive bond (e.g., carbon/hydrogen) is not available for bonding, the ultraviolet light-induced excitation of the benzophenone group is reversible and the molecule returns to ground state energy level upon removal of the energy source. Photoreactive aryl ketones such as benzophenone and acetophenone can undergo multiple reactivations in water and hence can provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benzenesulfonyl azide; and phosphoryl azides (RO)₂PON₃ such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetates (—CO—CN₂—CO—O—) such as t-butyl alpha diazoacetoacetate.

Other photoreactive groups include the diazirines (—CHN₂) such as 3-trifluoromethyl-3-phenyldiazirine; and ketenes (—CH═C═O) such as ketene and diphenylketene.

### Degradable Polymers

Degradable polymers can be used in conjunction with some embodiments herein. By way of example, degradable polymers can be included as a substrate, as a coating over a substrate, or as part of a polymeric matrix. Degradable polymers used with embodiments of the invention can include either natural or synthetic polymers. Examples of degradable polymers can include those with hydrolytically unstable linkages in the polymeric backbone. Degradable polymers of the invention can include both those with bulk erosion characteristics and those with surface erosion characteristics.

While not intending to be bound by theory, the use of degradable polyesters can be advantageous in the context of providing controlled release of nucleic acid delivery complexes because release can be mediated by degradation of the matrix in addition to diffusion through the matrix.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)—]x[—O—(CH₂)₄—O—C(O)—C₆H₄—C(O)—]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. The subscript “n” can be selected such that the molecular weight of the PEG block is between about 300 and about 4000. The repeating units “x” and “y” can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure. Such degradable polymers can specifically include those described in U.S. Pat. No. 5,980,948, the content of which is herein incorporated by reference in its entirety.

Degradable polyesteramides can include those formed from the monomers OH-x-OH, z, and COOH-y-COOH, wherein x is alkyl, y is alkyl, and z is leucine or phenylalanine. Such degradable polyesteramides can specifically include those described in U.S. Pat. No. 6,703,040, the content of which is herein incorporated by reference in its entirety.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

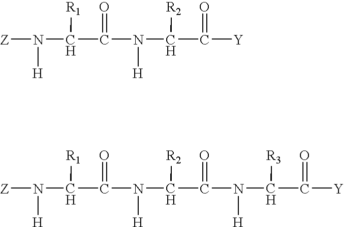

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, a aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Publ. Pat. Application No. 20070218102, entitled “HYDROPHOBIC DERIVATIVES OF NATURAL BIODEGRADABLE POLYSACCHARIDES”, all of which are herein incorporated by reference in their entirety.

Degradable polymers of the invention can also include dextran based polymers such as those described in U.S. Pat. No. 6,303,148, entitled “PROCESS FOR THE PREPARATION OF A CONTROLLED RELEASE SYSTEM”, the content of which is herein incorporated by reference in its entirety. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

### Non-Degradable Polymers

Non-degradable polymers can be used in conjunction with some embodiments herein. By way of example, non-degradable polymers can be included as a substrate, as a coating over a substrate, or as part of a polymeric matrix. In an embodiment, the non-degradable polymer includes a plurality of polymers, including a first polymer and a second polymer. When the coating solution contains only one polymer, it can be either a first or second polymer as described herein. As used herein, the term “(meth)acrylate”, when used in describing polymers, shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (PBMA) is used with a molecular weight of about 200,000 Daltons to about 300,000 Daltons.

Examples of suitable first polymers also include polymers selected from the group consisting of poly(aryl(meth)acrylates), poly(aralkyl(meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone, poly(methacryloxybenzotriazole), poly(naphthylacrylate) and -methacrylate), poly(4-nitrophenyl acrylate), and poly(phenyl acrylate) and -methacrylate). Examples of poly(aralkyl(meth)acrylates) include poly(benzyl acrylate) and -methacrylate), poly(2-phenethyl acrylate) and -methacrylate, and poly(1-pyrenylmethyl methacrylate). Examples of poly(aryloxyalkyl(meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. The pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), poly(ethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl(meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl(meth)acrylate (e.g., methyl methacrylate) and/or butene. Useful block copolymers include copolymer containing (a) blocks of polystyrene, (b) blocks of a polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Non-degradable polymers can also include those described in U.S. Publ. Pat. App. No. 2007/0026037, entitled “DEVICES, ARTICLES, COATINGS, AND METHODS FOR CONTROLLED ACTIVE AGENT RELEASE OR HEMOCOMPATIBILITY”, the contents of which are herein incorporated by reference in its entirety. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

### Negatively Charged Groups

Various embodiments of the invention include negatively charged groups on the surface of a material and/or within a matrix. The negatively charged groups can serve to electrostatically couple with nucleic acid delivery complexes. Exemplary negatively charged groups can include sulfates, sulfonates, carboxylates, phosphates, phosphonates, and the like.

In some embodiments, the quantity and/or density of negatively charged groups can be modulated in order to control the release rate/profile of nucleic acid delivery complexes from a device. By way of example, a given polymeric substrate or polymeric matrix may not include any negatively charged groups in its native unsubstituted form. However, through a chemical reaction the number of negatively charged groups can be changed. For example, additional negatively charged groups can be added to the polymer. As a specific example if a given polymer doesn't include negatively charged groups it can be combined as part of a copolymer with a component that does include charged groups. As another example, a negatively charged group can be added as a pendent group. As yet another example, charge density can be modified through pH modification.

In some embodiments, a polymer can be modified to include more or less negatively charged groups before the polymer is deposited to form a medical device. In other embodiments, a polymer can be modified to include more or less negatively charged groups after the polymer is deposited to form a medical device.

In various embodiments, modulation of charge density can be accomplished through modulation of the number of negatively charged groups on a polymer as described above.

### Substrates

In some embodiments, a substrate with negatively charged groups on the surface can be used. In some embodiments, a layer of a material that includes negatively charged groups can be disposed on the surface of a substrate. In still other embodiments, a matrix that includes negatively charged groups within the matrix and/or on the surface of the matrix can be disposed on a substrate.

Exemplary substrates can include metals, polymers, ceramics, and natural materials. Substrate polymers include those formed of synthetic polymers, including oligomers, homopolymers, and copolymers resulting from either addition or condensation polymerizations. Examples include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, styrene, vinyl chloride, vinyl acetate, vinyl pyrrolidone, and vinylidene difluoride, condensation polymers including, but are not limited to, polyamides such as polycaprolactam, polylauryl lactam, polyhexamethylene adipamide, and polyhexamethylene dodecanediamide, and also polyurethanes, polycarbonates, polysulfones, poly(ethylene terephthalate), polytetrafluoroethylene, polyethylene, polypropylene, polylactic acid, polyglycolic acid, polysiloxanes (silicones), cellulose, and polyetheretherketone.

Embodiments of the invention can also include the use of ceramics as a substrate. Ceramics include, but are not limited to, silicon nitride, silicon carbide, zirconia, and alumina, as well as glass, silica, and sapphire.

Substrate metals can include, but are not limited to, cobalt, chromium, nickel, titanium, tantalum, iridium, tungsten and alloys such as stainless steel, nitinol or cobalt chromium. Suitable metals can also include the noble metals such as gold, silver, copper, platinum, and alloys including the same.

In some embodiments, a tie layer can be disposed over a substrate, such as a metal, in order to facilitate the attachment of other components thereon. By way of example, a tie layer (such as a silane compound with —C—H groups) can be deposited on a metal, then a polymer with negatively charged groups can be bonded to the tie layer through the reaction product of a photoreactive group on the polymer and —C—H groups on the tie layer.

Certain natural materials can also be used in some embodiments including human tissue, when used as a component of a device, such as bone, cartilage, skin and enamel; and other organic materials such as wood, cellulose, compressed carbon, rubber, silk, wool, and cotton. Substrates can also include carbon fiber. Substrates can also include resins, polysaccharides, silicon, or silica-based materials, glass, films, gels, and membranes.

### Medical Devices

Embodiments of the invention can include and can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

In some aspects, embodiments of the invention can include and be utilized in conjunction with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in conjunction with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. No. 6,719,750 B2 (“Devices for Intraocular Drug Delivery,” Varner et al.) and U.S. Pat. No. 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. No. 11/204,195 (filed Aug. 15, 2005, Anderson et al.), Ser. No. 11/204,271 (filed Aug. 15, 2005, Anderson et al.), Ser. No. 11/203,981 (filed Aug. 15, 2005, Anderson et al.), Ser. No. 11/203,879 (filed Aug. 15, 2005, Anderson et al.), Ser. No. 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

It will be appreciated that embodiments of the invention can also be used without substrates. By way of example, embodiments can include a matrix with nucleic acid delivery complexes disposed therein in the form of a filament or other shape without including a substrate.

It will be appreciated that embodiments herein can include devices, such as medical devices, that include a surface or a portion of a matrix that includes negatively charged groups and a surface or a portion of a matrix that does not include negatively charged groups. In this manner, the portion of the device which serves to provide release of the nucleic acid delivery complexes can be selectively chosen for particular applications.

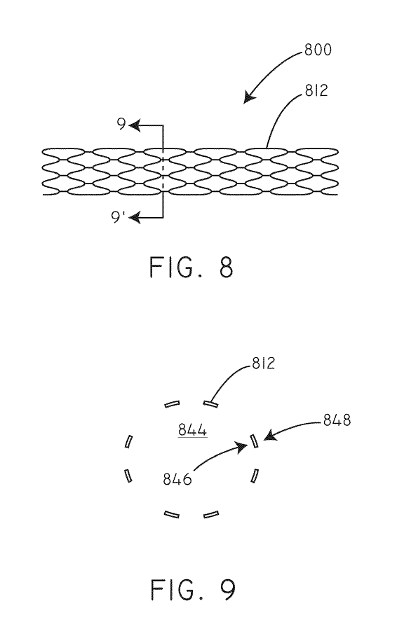

Referring to FIG. 8, a schematic view of an exemplary medical device 800 is shown. The medical device 800 can include struts (or wires) 812. FIG. 9 is a schematic cross-sectional view of the medical device 800 as taken along line 9-9′ of FIG. 8. As shown, the medical device 800 defines a central lumen 844. The surfaces of the medical device facing the central lumen 844 can be referred to as the luminal surface 846. The surfaces facing away from the central lumen can be referred to as the abluminal surface 848. In the context of a medical device that is a stent, when deployed in vivo, the abluminal surfaces 848 will generally be in contact with a vessel wall while the luminal surfaces 846 will generally interface with a stream of blood. In some applications, it can be desirable to administer an active agent to the vessel wall while limiting the amount that is released into the passing flow of blood. As such, in some embodiments, the abluminal surface 848 of the medical device can be made to include negatively charged groups to be able to hold and then release nucleic acid delivery complexes while the luminal surface 846 can be substantially free of negatively charged groups and therefore substantially free of nucleic acid delivery complexes. It will be appreciated that this serves as merely one specific example of a medical device including surfaces with negatively charged groups and surfaces without negatively charged groups and that many other specific devices and configurations are included within the scope herein.

The present invention may be better understood with reference to the following examples. These examples are intended to be representative of specific embodiments of the invention, and are not intended as limiting the scope of the invention.

### EXAMPLES

### Example 1

### Preparation of 4-Benzoylbenzoyl Chloride (BBA-Cl)

BBA-Cl was prepared in accordance with the method described in Example 1 of U.S. Pat. No. 7,056,533, the content of which is herein incorporated by reference in its entirety.

### Example 2

### Preparation of N-(3-Aminopropyl)methacrvlamide Hydrochloride (APMA)

APMA was prepared in accordance with the method described in Example 2 of U.S. Pat. No. 7,056,533, the content of which is herein incorporated by reference in its entirety.

### Example 3

### Preparation of N-[3-(4-Benzoylbenzamido)propyl]methacrvlamide (BBA-APMA)

BBA-APMA was prepared in accordance with the method described in Example 3 of U.S. Pat. No. 7,056,533, the content of which is herein incorporated by reference in its entirety.

### Example 4

### Preparation of Polyacrylamide(36%)co-Methacrylic acid(MA)-(10%)co-Methoxy PEG 1000MA-(4%)co-BBA-APMA (PA-MA-PEG-BBA-APMA)

PA-MA-PEG-BBA-APMA was prepared in accordance with the method described in Example 4 of U.S. Pat. No. 7,056,533, the content of which is herein incorporated by reference in its entirety.

### Example 5

### Preparation of Photo-PA-AMPS

A copolymer of 93% acrylamide, 5% 2-acrylamide-2-methylpropanesulfonic acid (“AMPS”), and 2% N-(3-aminopropyl)methacrylamide (“APMA”) was prepared. Copolymerization of the acrylamide, AMPS, and APMA was followed by photoderivatization of the copolymer using 4-benzoylbenzoyl chloride under Schotten-Baumann conditions. The resulting copolymer with a photoactivatable group is referred to herein as Photo-PA-AMPS.

### Example 6

### Preparation of Photo-PVP

A copolymer of 99% N-vinylpyrrolidone and 1% N-(3-aminopropyl)methacrylamide (APMA) was prepared. Copolymerization of 1-vinyl-2-pyrrolidone and APMA was followed by photoderivatization of the copolymer using 4-benzoylbenzoyl chloride under Schotten-Baumann conditions. The resulting copolymer with a photoactivatable group is referred to herein as Photo-PVP.

### Example 7

### Preparation of Photo-Heparin

A photoreactive derivative of heparin (photo-heparin) was prepared by reacting heparin with benzoyl-benzoyl-epsilon-aminocaproyl-N-oxysuccinimide in dimethylsulfoxide/carbonate buffer, pH 9.0. The solvent was evaporated and the photoheparin was dialyzed against water, and lyophilized, and then dissolved in water at 3 mg/ml. The product can also be referred to as BBA-EAC-heparin (referring to the benzophenone photoreactive group benzoyl benzoic acid, BBA; and the spacer, epsilon aminocaproic acid, EAC).

### Example 8

### Attachment of Nucleic Acid Delivery Complexes to Surfaces and Transfection Efficiency

Photopolymer solutions (polymers with photoreactive groups) were prepared at 2 or 10 mg/ml in H₂O and then sterile filtered. Specifically, seven different photopolymer solutions were prepared as shown in Table 1 below.

TABLE 1

| Solution | | | | Negatively |
|---|---|---|---|---|
| ID | Photopolymer | Concentration | | Charged Groups |
| 1 | Photo-PA-AMPS | 2 | mg/ml | Sulfonate |
| 2 | Photo-PA-AMPS | 10 | mg/ml | Sulfonate |
| 3 | Photo-Heparin | 2 | mg/ml | Sulfate |
| 4 | Photo-Heparin | 10 | mg/ml | Sulfate |
| 5 | Photo-PVP | 2 | mg/ml | None |
| 6 | Photo-PVP | 10 | mg/ml | None |
| 7 | PA-MA-PEG-BBA-APMA | 10 | mg/ml | Carboxylate |

The photopolymer solutions were then added to the wells of a 96 well tissue culture polystyrene (TCPS) plate. 100 μl of a photopolymer solution or pure H₂O (control) were then added to each well tested. The 96 well plate was then exposed to broad spectrum UV light (Dymax PC-2 UV Light Source) for 3 minutes.

Plasmid DNA encoding green fluorescent protein (gWiz-GFP) was obtained from Aldevron (Fargo, N. Dak.). The plasmid DNA was then labeled with Cy3 (fluorophore) according to labeling kit instructions (Mirus Bio, Madison, Wis.). A solution of nucleic acid delivery complexes was formed by combining equal volumes of H₂O containing Cy3 labeled plasmid DNA and PEI (polyethylenimine, branched, 25 kDa, Sigma-Aldrich, St. Louis, Mo.) resulting in a final concentration of 10 μg/ml DNA and 6 μg/ml PEI to give an N:P ratio of 5.

The wells of the 96 well plate were rinsed 3 times in H₂O. Then 100 μl of the nucleic acid delivery complex solution was added to each well and incubated overnight.

After removal of nucleic acid delivery complex solution the amount of Cy3 fluorescence remaining in solution was measured to determine the amount of DNA which was bound to the surface of the wells.

As shown in FIG. 4, PEI/DNA complexes preferentially bound to all polymers with negatively charged groups as compared to the uncharged TCPS surface. A greater degree of binding was seen with Photo-PA-AMPS and Photo-Heparin modified surfaces. Positively charged nucleic acid delivery complexes were expected to associate with these polymers through electrostatic interactions due to the presence of negatively charged sulfate groups on Photo-PA-AMPS and Photo-Heparin.

HEK-293 cells (ATCC, Manassas, Va.) were then added to plates at a density of 10,000 cells/well and cultured for 72 hours. Cells were then lysed and GFP protein levels were measured using a fluorescent plate reader.

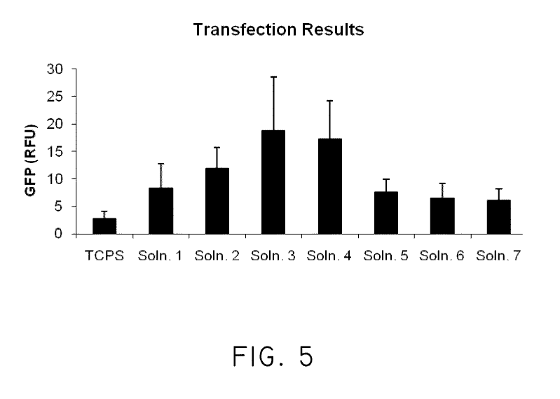

The transfection results are shown in FIG. 5. Elevated levels of GFP expression were seen for cells plated on all surfaces modified with photopolymers as compared to TCPS. Corresponding to the DNA coating data, the highest levels of GFP expression were seen in cells cultured on Photo-PA-AMPS and Photo-Heparin modified surfaces.

### Example 9

### Controlled Release of PEI/DNA Complexes From Surfaces

Photopolymer solutions were prepared at various concentrations in H₂O and then sterile filtered. Specifically, seven different photopolymer solutions were prepared as shown in Table 1 below.

TABLE 2

| Solution | | | | Negatively |
|---|---|---|---|---|
| ID | Photopolymer | Concentration | | Charged Groups |
| 8 | Photo-Heparin | 1 | mg/ml | Sulfate |
| 9 | Photo-Heparin | 5 | mg/ml | Sulfate |
| 10 | Photo-Heparin | 10 | mg/ml | Sulfate |
| 11 | Photo-PA-AMPS | 1 | mg/ml | Sulfonate |
| 12 | Photo-PA-AMPS | 5 | mg/ml | Sulfonate |
| 13 | Photo-PA-AMPS | 10 | mg/ml | Sulfonate |
| 14 | Photo-PVP | 10 | mg/ml | None |

The photopolymer solutions were then added to the wells of a 96 well tissue culture polystyrene (TCPS) plate. 100 μl of a photopolymer solution or pure H₂O (control) were then added to each well tested. The 96 well plate was then exposed to broad spectrum UV light (Dymax PC-2 UV Light Source) for 3 minutes.

A PEI/DNA complex solution was formed by combining equal volumes of H₂O containing Cy3 labeled DNA (labeled according to manufacturer's instructions) and PEI resulting in a final concentration of 10 μg/ml DNA and 6 μg/ml PEI to give an N:P ratio of 5.

The wells of the 96 well plate were rinsed 3 times in H₂O. Then 100 μl of the PEI/DNA solution was added to each well and incubated for 24 hours and then removed. Phosphate buffered saline (PBS) was added to wells and was replaced with fresh PBS at 2, 6, 24, 48 and 120 hours. The amount of DNA eluted into PBS was determined using a fluorescence plate reader with filters appropriate for Cy3 detection.

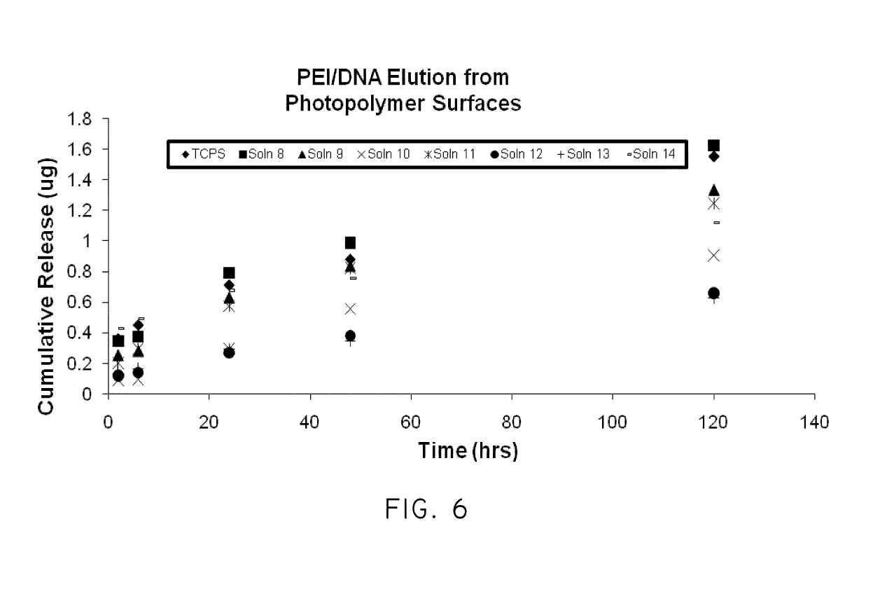

Elution results are shown in FIG. 6. Elution of nucleic acid delivery complexes was seen from all surfaces including TCPS. All polymers tested demonstrated delayed release of complex as compared to TCPS. There was a concentration dependent effect on release rates seen for both Photo-Heparin and Photo-PA-AMPS for which increasing concentrations resulted in reduced amounts of released PEI/DNA. At equivalent concentrations a larger amount of nucleic acid delivery complexes were released from Photo-Heparin surfaces than Photo-PA-AMPS surfaces.

### Example 10

### Complex Retains Activity after Mixing with Photo-PA-AMPS

HR5CL11 cells (European Collection of Cell Cultures, Salisbury, UK) were obtain. HR5CL11 cells express luciferase under the control of a doxycycline inducible promoter. The cells were cultured in Dulbecco's Modified Eagle Medium (DMEM, Invitrogen, Carlsbad, Calif.) with 10% fetal bovine serum (FBS).

siRNA targeting luciferase or scrambled controls were obtained from Qiagen (Valencia, Calif.). NTER, a cationic peptide siRNA delivery vehicle was obtained from Sigma (St. Louis, Mo.). Lipofectamine RNAiMax, a cationic lipid siRNA delivery vehicle was obtained from Invitrogen (Carlsbad, Calif.).

One day before transfection, the HR5CL11 cells were plated in 96 well plates at 10,000 cells per well. On the day of transfection, siRNA was complexed with NTER and Lipofectamine RNAiMax as per manufacturer's instructions resulting in siRNA complexes at a 650 nM concentration of siRNA.

Photo-PA-AMPS(PPA) was dissolved in water at 1 or 10 mg/ml. 5 μl of Lipofectamine complexes were mixed with 5 μl of Photo-PA-AMPS solution or a water only control and incubated for 15 minutes. 15 μl of NTER complexes were mixed with 5 μl of Photo-PA-AMPS solution or a water only control and incubated for 15 minutes. Complexes were then diluted in DMEM with 10% FBS and 5 μg/ml doxycycline (Sigma) to final siRNA concentrations of 30 nM for NTER complexes and 10 nM for Lipofectamine complexes.

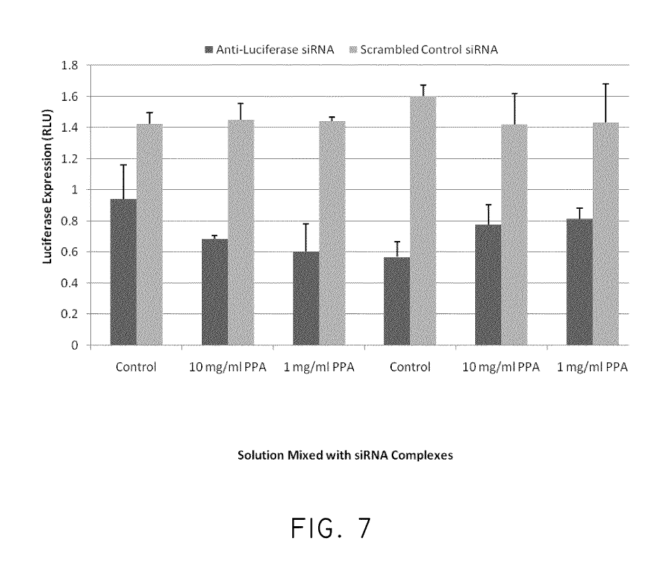

Media was removed from the cells and replaced with 100 μl of media containing siRNA complexes, Photo-PA-AMPS at a final concentration of 17 or 170 μg/ml, 10% FBS and 5 μg/ml doxycycline. Cells were cultured for 24 hours and then gene knockdown was assessed by measuring luciferase levels. Cells were lysed in 50 μl Glo Lysis Buffer (Promega, Madison, Wis.) and lysates were mixed with 50 μl of Bright Glo Luciferase Reagent and luciferase expression as indicated by luminescence was measured on a plate luminometer. Results (mean+/−standard deviation of triplicate samples) are shown in FIG. 7. It can be seen that transfection efficiency of both cationic peptides and cationic lipids is not inhibited by Photo-PA-AMPS at 1 or 10 mg/ml concentrations.

It should be noted that, as used in this specification the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference. Nothing herein is to be construed as an admission that the inventors are not entitled to antedate any publication and/or patent, including any publication and/or patent cited herein.

The invention has been described with reference to various specific and preferred embodiments and techniques. However, it should be understood that many variations and modifications may be made while remaining within the spirit and scope of the invention.

### Further Embodiments

In an embodiment the invention includes a medical device including a substrate surface, a polymeric coating disposed on the surface, the polymeric coating coupled to the substrate surface through the reaction product of a photoreactive group, the polymeric coating comprising negatively charged species on the surface, and a plurality of nucleic acid delivery complexes disposed on the polymeric coating, the nucleic acid delivery complexes comprising a nucleic acid and a cationic carrier agent complexed to the nucleic acid.

In an embodiment, the photoactivable moiety includes a benzophenone group. In an embodiment, the negatively charged species can be selected from the group consisting of carboxylates, sulfonates, sulfates, phosphonates and phosphates. In an embodiment, the negatively charged species can be selected from the group consisting of sulfonates and sulfates. In an embodiment, the negatively charged species can have a pKa of less than about 6. In an embodiment, the negatively charged species can have a negative charge in an aqueous solution at a pH of between about 6.8 and 7.4. In an embodiment, the polymeric coating includes hydrolytically labile bonds configured to break down in vivo. In an embodiment, the plurality of nucleic acid delivery complexes electrostatically bonded to the negatively charged species on the surface of the polymeric coating. In an embodiment, the cationic carrier agent selected from the group consisting of cationic polymers, cationic lipids, and cationic dendrimers. In an embodiment, the cationic carrier agent can include PEI.

In an embodiment the invention includes a medical device including a polymer bound to a surface, the polymer having a net negative charge in an aqueous solution, a plurality of nucleic acid delivery complexes disposed on the surface of the polymer, the nucleic acid delivery complexes electrostatically held to the surface of the polymer, the polymer coupled to a substrate surface through the reaction product of a photoreactive group. In an embodiment, the polymer can have a net negative charge in an aqueous solution at a pH of between 6.0 and 8.0.

In an embodiment, the invention can include a medical device including a polymeric matrix comprising a polymer with anionic groups, a plurality of nucleic acid delivery complexes disposed within the polymeric matrix, the nucleic acid delivery complexes including a nucleic acid and a carrier agent complexed to the nucleic acid.

In an embodiment, the invention includes a method of depositing a coating with an active agent on a medical device including depositing a polymer solution onto a substrate to form a coated substrate, the polymer solution comprising a polymer with negatively charged species and photoreactive groups, applying actinic radiation to the coated substrate, contacting the coated substrate with nucleic acid delivery complexes, the nucleic acid delivery complexes comprising a nucleic acid and a carrier agent complexed to the nucleic acid. In an embodiment, the substrate can have a surface onto which the polymer solution is applied, the substrate surface comprising C—H bonds. In an embodiment, the substrate comprising a bonding layer, the bonding layer comprising C—H bonds.

In an embodiment, the invention includes a method of making an implantable medical device including depositing a polymer composition, modulating the density of negatively charged groups on the polymer composition, and disposing nucleic acid delivery complexes on the polymer composition. The nucleic acid delivery complexes can include a nucleic acid and a cationic carrier agent complexed to the nucleic acid. Modulation of the density of negatively charged groups on the polymer composition results in control of the release of the nucleic acid delivery complexes from the polymer composition.

In an embodiment, the invention includes a method of controlling the release rate of nucleic acid delivery complexes including modulating the charge density of a drug delivery matrix, the drug delivery matrix comprising a polymer and nucleic acid delivery complexes electrostatically held to the drug delivery matrix. In an embodiment, modulating comprises increasing or decreasing the charge density. In an embodiment, modulating comprises adding anionic groups to the polymer of the drug delivery matrix.

In an embodiment, the invention includes a method of providing controlled release of a nucleic acid including selecting a desired release profile and modulating the charge density of a drug delivery matrix according to the desired release profile. In an embodiment, modulating comprises increasing or decreasing the number of negatively charged groups. In an embodiment, modulating includes adding negatively charged groups to the polymer of the drug delivery matrix.

Embodiments of the invention can also include kits including one or more of the components described herein.

1. A medical device comprising:
a substrate surface;
a polymeric coating disposed on the surface, the polymeric coating coupled to the substrate surface through the reaction product of a photoreactive group; the polymeric coating comprising negatively charged species on the surface; and
a plurality of nucleic acid delivery complexes disposed on the polymeric coating, the nucleic acid delivery complexes comprising a nucleic acid and a cationic carrier agent complexed to the nucleic acid.

2. The medical device of claim 1, the photoactivable moiety comprising a benzophenone group.

3. The medical device of claim 1, the negatively charged species selected from the group consisting of carboxylates, sulfonates, sulfates, phosphonates and phosphates.

4. The medical device of claim 1, the negatively charged species selected from the group consisting of sulfonates and sulfates.

5. The medical device of claim 1, the negatively charged species having a pKa of less than about 6.

6. The medical device of claim 1, the negatively charged species having a negative charge in an aqueous solution at a pH of between about 6.0 and 8.0.

7. The medical device of claim 1, the polymeric coating including hydrolytically or enzymatically labile bonds configured to break down in vivo.

8. The medical device of claim 1, the plurality of nucleic acid delivery complexes electrostatically bonded to the negatively charged species on the surface of the polymeric coating.

9. The medical device of claim 1, the cationic carrier agent selected from the group consisting of cationic polymers, cationic lipids, and cationic peptides.

10. The medical device of claim 1, the cationic carrier agent comprising PEI.

11. The medical device of claim 1, the nucleic acid selected from the group consisting of RNA, DNA, miRNA, piRNA, siRNA, shRNA, antisense nucleic acids, aptamers, ribozymes, and catalytic DNA.

12. The medical device of claim 1, the polymeric coating comprising a photo-derivatized copolymer of acrylamide, 2-acrylamide-2-methylpropanesulfonic acid, and N-(3-aminopropyl)methacrylamide.

13. The medical device of claim 1, the medical device including a surface that is substantially free of negatively charged species.

14. A medical device comprising:
a polymeric matrix comprising a polymer with anionic groups;
a plurality of nucleic acid delivery complexes disposed within the polymeric matrix, the nucleic acid delivery complexes comprising a nucleic acid and a carrier agent complexed to the nucleic acid.

15. The medical device of claim 14, the polymer with anionic groups comprising a polysaccharide based polymer.

16. The medical device of claim 14, the polymeric matrix further comprising a second polymer.

17. The medical device of claim 14, the second polymer comprising a polysaccharide based polymer.

18. A method of controlling the release rate of nucleic acid delivery complexes comprising:
modulating the charge density of a drug delivery matrix, the drug delivery matrix comprising a polymer and nucleic acid delivery complexes electrostatically held to the drug delivery matrix.

19. The method of claim 18, wherein modulating comprises increasing or decreasing the charge density.

20. The method of claim 18, wherein modulating comprises adding anionic groups to the polymer of the drug delivery matrix.

21. The method of claim 18, wherein the nucleic acid delivery complexes retain activity in vivo after being released from the drug delivery matrix.

22. The method of claim 18, wherein the nucleic acid delivery complexes are electrostatically held to the surface of the drug delivery matrix.

23. The method of claim 18, wherein the nucleic acid delivery complexes are electrostatically held within the drug delivery matrix.
